(19) United States
(12) Patent Application Publication
(10) Pub. No.: US 2025/0090150 A1
(43) Pub. Date: Mar. 20, 2025

(54) HANDLE ASSEMBLY FOR EXPANDABLE CARRIAGE FOR JOINTLY CLAMPING EXTREMITY HOLDER AND TRACK OF BASE PLATE

(71) Applicant: Innovative Medical Products, Inc., Plainville, CT (US)

(72) Inventor: Tamas KOVACS, Burlington, CT (US)

(73) Assignee: Innovative Medical Products, Inc., Plainville, CT (US)

(21) Appl. No.: 18/960,901

(22) Filed: Nov. 26, 2024

Related U.S. Application Data
(63) Continuation-in-part of application No. 17/187,759, filed on Feb. 27, 2021, now Pat. No. 12,150,818.

(51) Int. Cl. A61B 17/00

(52) U.S. Cl. CPC A61B 17/00; A61B 2017/00464

(57) ABSTRACT

A carriage assembly is disclosed for quasi-independent, multi-action clamping of multiple surgical components such as a track of a base plate and a ball of an extremity holder. Clamping is achieved via tightening a single handle for adjustably positioning an extremity holder for a body part during a surgical procedure. When the handle assembly is tightened, in a first action, a first tab clamp portion of a tab couples to the carriage body while moving the carriage arm toward the carriage body to thereby clamp a first surgical device. In a second action, the tab pivots about the first tab clamp portion in the Y direction, to thereby clamp a second surgical device in between the second tab clamp portion and the track portion of the first body, to achieve simultaneous, multi-action clamping. The invention solves the problems of sterilizability, wear of surgical equipment, precise holding, and ease of positioning/repositioning.

### CROSS-REFERENCE TO RELATED APPLICATION

This application is a continuation-in-part of, and claims the benefit of, co-pending U.S. patent application Ser. No. 17/187,759 filed Feb. 27, 2021, now U.S. Pat. No. 12,150,818 B2, entitled “Expandable Carriage with Adjustable Cross Nut,” that is incorporated by reference in its entirety herein.

### FIELD OF THE INVENTION

The present invention relates to an apparatus of the type used for the positive positioning of a body part for surgical procedures and other medical activities where the body part must be maintained in the selected optimum position with respect to an operating room table. In particular, this invention relates to a patient positioning apparatus positionable by one hand, having improved holding characteristics, and sterilization features.

### BACKGROUND OF THE INVENTION

In a first problem, medical devices within the sterile surgical field need to be sterilized, between one surgery and the next. Medical positioning systems for holding a body part, including surgical equipment, invariably come in contact with body tissues or fluids, and are therefore considered critical items. These items need to be sterile when used, because any microbial contamination could result in disease transmission. Consequently, there is a need for surgical positioning systems to reduce, or eliminate, features such as crevices that collect and retain body tissues or fluids, as well as to reduce the number of parts to improve sterilization and reduce disease transmission.

If these items are heat resistant, the recommended sterilization process is steam sterilization, because it has the largest margin of safety due to its reliability, consistency, and lethality. Most medical and surgical devices used in healthcare facilities are made of materials that are heat stable and therefore undergo heat, primarily steam, sterilization. Sterilization destroys all microorganisms on the surface of an article or in a fluid to prevent disease transmission associated with the use of that item. Sterilization refers to any process that eliminates, removes and/or kills all forms of life, including transmissible agents such as fungi, bacteria, viruses, spore forms, etc., present on a surface, which can be achieved by applying heat, chemicals, irradiation, high pressure, and filtration or combinations thereof. The concept of what constitutes “sterile” is measured as a probability of sterility for each item to be sterilized. This probability is commonly referred to as the sterility assurance level (SAL).

Sterilization facilities, such as Sterile Processing Departments (SPDs) are located separately from operating rooms, as these facilities perform heat sterilization on surgical equipment in an autoclave (sometimes called a converter). If, however, sterilization is needed in the operating room, flash sterilization methods may be used; for example, should the surgical apparatus become unsterile for some reason, then flash sterilization methods require placing the item in hot liquids to remove pathogens. After flash sterilization occurs, the surgical apparatus may take longer to cool down and reach the required room-temperature, which, in turn, delays the surgical procedure and adds cost.

Problems in sterilizing surgical equipment for holding a limb, joint, or other body part abound. Disadvantages of known surgical equipment include the costs associated with sterilization, whereby nooks and folds where residues accumulate and cannot be, or require increased effort to be, dislodged. Another design problem is in use of multiple parts, whereby the entire set of complex rails, tubes and other parts of the surgical equipment and apparatus required disassembly, scrubbing, power washing and other procedures that increased the cost of the sterilization and overall procedure. In addition, parts of the surgical equipment for holding a limb, joint, or other body part could get lost, or removal may void warranties. In general, such surgical equipment requires sterilization before entering an operating room. Once the surgical procedure ends, the surgical equipment again requires sterilization.

A second problem involves the wear of clamping components associated with a surgical apparatus as it is used over time. In the field of this invention, a clamping assembly typically achieves a clamping effect by way of external and internal threads, such as a screw-type assembly. The wearing of clamping components has been shown to be particularly prevalent for these threaded components.

FIGS. 1A and 1B provide schematic views of an illustrative screw-type assembly that exhibits no signs of wear in its present condition. FIG. 1B is a schematic sectional view of FIG. 1A, taken along section A-A. The illustrative schematic of FIGS. 1A and 1B show a new, unworn screw-type assembly includes external threads 200 and internal threads 210 that run along a common axis 220. External and internal threads 200, 210 may be characterized by conventional thread geometry including, but not necessarily limited to, a root 230, a crest 240, a flank 250, a pitch 260, a minor diameter 270, a major diameter 280, and a thread angle α. As defined here, one or more of these parameters are shared by the external and internal thread components 200, 210, albeit subject to any tolerance(s) that allow proper coupling of the screw-type system. Of particular importance to the present application are external start 290a and internal start 290b. As shown in FIG. 1B, internal start 290b is defined at a first radial position; upon threading external threads 200 into internal threads 210, external start 290a will meet internal start 290b at the first radial position to commence the threading process.

FIGS. 1C and 1D provide analogous schematic views of the same illustrative screw-type assembly; however, this system has undergone some amount of plastic deformation and, as a result, shows signs of wear in its present condition. Here, FIG. 1D provides a schematic sectional view of FIG. 1C, taken along section B-B. The illustrative schematic of FIGS. 1C and 1D show a used screw type-assembly that is primarily characterized by a change in radial location of internal start 290b. This parameter is denoted as internal start 290b′ and is offset radially from the first radial position of internal start 290b by a deformation angle θ, as shown in FIG. 1D. The used/worn system of FIGS. 1C and 1D may exhibit changes in geometry to at least one or more of the parameters that define internal threads 210, which may include a root 230′, a crest 240′, a flank 250′, a pitch 260′, and a thread angle α′.

Internal threads 210 in particular are subject to wear that manifests as plastic deformation of its components, including, but not limited to, the radial position of the internal start 290b. This plastic deformation results in localized changes along internal threads 210. External threads 200 are subject to similar wear, although the associated plastic deformation has been found to be less prevalent for external thread components. Such clamping assemblies typically include a handle which is used to tighten the clamp assembly to achieve the aforementioned maintained positive positioning of the overall surgical apparatus. This wear on the threaded components changes the angular positioning of the handle-particularly when the handle is in the tightened position (that which achieves maintained positive positioning of the overall surgical apparatus). The angular positioning of the handle is important; improper angular positioning at a minimum makes maintaining, releasing, and repositioning more difficult for the surgeon to achieve. Improper angular positioning may cause more drastic problems, such as when the handle abuts nearby components of the surgical apparatus (e.g., the base plate).

A third problem involves precise holding of the patient in a particular position. Surgical procedures increasingly require precise and predictable positioning of the joint, limb, or other body part corresponding to the surgical site. The positioner must be able to hold under the static weight of the apparatus and the weight of the patient. The positioner must also be able to hold under applied external forces that typically act upon the patient in a manner that may unintentionally move the patient during the surgery.

A fourth problem involves ease of positioning, and repositioning, by the surgeon. Optimal positioning requires a large range of easily available positive positioning adjustments, so that the limb, joint, or other body part can be precisely positioned and thereafter maintained in that desired position. While precisely-maintainable positioning is important, so too is the ability to release and reposition the limb, joint, or other body part on demand during the course of the procedure. The surgeon may, for example, desire to position or reposition a clamping device with a single hand, where the surgeon's other hand is holding something else. The physical space around a surgical site may limit the number of surgical staff that can access certain areas, and for at least these reasons, it is desirable to provide an apparatus that is easily manipulable. Therefore, an apparatus, system, and method that provides maintainable, releasable, and repositionable positive positioning ensures optimal access to surgical sites, which require a variety of angular relationships for effective surgery.

These problems of sterilizability, wear of surgical equipment, precise static holding, and ease of dynamic positioning and/or repositioning—and other problems that will become clear in reading this disclosure—have existed for a long period of time without an optimal solution. These problems also involve design and construction involving multiple parts such as, for example, complex rails, tubes and other apparatus secured to an operating table. As a result, a simplified design for surgical equipment for holding a limb, joint, or other body part is desired that reduces these and other disadvantages including the cost of the sterilization and overall time and cost of the procedure.

### SUMMARY OF THE INVENTION

A first object of the apparatus, system, and method of the present invention is to provide a carriage with a minimum number of reentrant surfaces and with clearances between parts to allow a spray sterilizer to access adjacent mated parts. Reentrant surfaces in this context possess an overhang or bend back on themselves, thus limiting the line-of-sight of the cleaning mechanism resulting in reduced sterilizability. Components of the systems disclosed herein may provide 5 to 10 thousandths of an inch clearance, which provides adequate space for the sterilizer spraying mechanism, and other cleaning mechanisms, to access those reentrant surfaces to adequately flush through and sterilize them.

The first object of the apparatus, system and method of the present invention may be to provide a carriage having advantages of minimum parts so as to improve use, sterilization, maintenance thereof.

A second object of the apparatus, system, and method of the present invention is to provide a clamp assembly with an ability to adjust the clocking of the handle as the associated threaded components wear over time. In one embodiment, the clocking of the handle may be adjusted by providing washers of varying thicknesses that are interchangeable as wear occurs; the invention may employ an external retainer ring designed to snap on and only be removed by destroying it, which provides the added benefit of keeping the components together so that they aren't lost in the hospital, in between surgeries. In another embodiment, the present invention may provide an adjustable cross nut to achieve modifiable clocking. In yet another embodiment, an adjustable handle may be employed, such as one that may employ a spring-loaded mechanism, and which adjusts the handle and its position on the thread rod to which it is attached via a fastener such as a knurled knob adjustable by hand or screw driver; the adjustable handle may further include a plurality of teeth on the handle, and corresponding teeth on the rod, such that by loosening the fastener an angular adjustment of the handle may be achieved relative to the rod, and subsequently retightened.

A third object of the apparatus, system, and method of the present invention is to provide a carriage for positioning an extremity holder featuring two clamping actions achieved by a single locking mechanism. In a first clamping action, the carriage may fixedly couple to an extremity holder that is, in turn, coupled to a body part of a patient. The first clamping action may employ a hinge disposed in a predetermined location that increases the clamping force, i.e., holding power, of the extremity holder by virtue of the static friction resulting from shear direction movement-which may invoke Van der Waals bonding. In a second clamping action, the carriage may fixedly couple to a track of a baseplate that is, in turn, coupled to the side rail of an operating table. In the second clamping action, the carriage may alternatively fixedly couple to the side rail of an operating table, or other component.

In the third object of the apparatus, system and method, the present invention may provide an extremity holder, such as for a positioning a knee for surgery, utilizing a locking the ball and carriage with a one arm lock and ergonomic handle, where the handle maintains an optimal angular position upon tightening; such as, for example, between approximately a twelve-o'clock and approximately a three-o'clock position.

A fourth object of the apparatus, system, and method of the present invention provide a carriage having a track clamp that achieves a quasi-independent, equalizing clamping action with respect to two coupled components to be clamped thereto, such as an extremity holder and a track of a baseplate, wherein the extremity holder may be configured to couple to the limb of a patient for surgical operation, and wherein the track of the baseplate may be configured to couple to a side rail of an operating room or support table. The equalizing action may be configured to first lock the extremity holder in place so that the carriage may be further slidably adjusted along the track and then, upon further tightening of the handle, lock the carriage with respect to the operating table.

A fifth object of the apparatus, system, and method of the present invention is to provide a carriage having a track clamp and a clamp assembly with the necessary clearance for the clamp assembly to tilt laterally with respect to the arm of the carriage to catch and line up with the threaded opening as it approaches an aligned position of the arm from opened to closed position.

A sixth object of the apparatus, system, and method of the present invention may be to provide a carriage having a flip handle designed to flip in a manner that allows for approximately 180 degree of adjustable rotation and that may have one or more discrete locking positions, in the flip direction, to keep the handle from flopping around while the surgeon is making adjustments.

Other desirable features and characteristics will become apparent from the subsequent detailed description, the drawings, and the appended claims, when considered in view of this background.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1A illustrates a schematic view of a single-start screw-type assembly exhibiting no signs of wear, in accordance with an embodiment of the present invention;

FIG. 1B illustrates a schematic sectional view of the single-start screw-type assembly of FIG. 1A, taken along section A-A, in accordance with an embodiment of the present invention;

FIG. 1C illustrates a schematic view of a single-start thread design, which has undergone plastic deformation resulting from wear, in accordance with an embodiment of the present invention;

FIG. 1D illustrates a schematic sectional view of the single-start screw-type assembly of FIG. 1C, taken along section B-B, in accordance with an embodiment of the present invention;

FIG. 2 illustrates a perspective view of a carriage and an adjustable cross nut assembly, an extremity holder, a baseplate having a track, and a clamp disposed on a conventional operating table having side rails, in accordance with an embodiment of the present invention;

FIG. 3 illustrates a top, prospective, partially-exploded view of the apparatus, system, and method in accordance with an embodiment of the present invention;

FIG. 4 illustrates a top, partially-exploded view of the apparatus in accordance with an embodiment of the present invention;

FIG. 5 illustrates a bottom, partially-exploded view the apparatus in accordance with an embodiment of the present invention;

FIG. 6 illustrates a side, partially-exploded view of the apparatus in accordance with an embodiment of the present invention;

FIG. 7 illustrates another side, partially-exploded view of the apparatus in accordance with an embodiment of the present invention;

FIG. 8 illustrates another side, partially-exploded view of the apparatus in accordance with an embodiment of the present invention;

FIG. 9 illustrates another side, partially-exploded view of the apparatus in accordance with an embodiment of the present invention;

FIG. 10A illustrates a perspective view of an adjustable cross nut assembly in accordance with an embodiment of the present invention;

FIG. 10B illustrates a side view of an adjustable cross nut assembly in accordance with an embodiment of the present invention;

FIG. 11 illustrates a perspective view of a clamp assembly according to an embodiment of the present invention;

FIG. 12 illustrates a perspective view of a carriage according to an embodiment of the present invention;

FIG. 13A illustrates a side view of part of a carriage according to an embodiment of the present invention;

FIG. 13B illustrates a partial side view of part of a carriage and an adjustable cross nut assembly according to an embodiment of the present invention;

FIGS. 14A, 14B, and 14C illustrate top views of an adjustable cross nut assembly inserted into a carriage at different depths, according to an embodiment of the present invention;

FIGS. 15A, 15B, 15C, 15D, 15E, and 15F illustrate perspective, partially-exploded views of a method of assembling a carriage and an adjustable cross nut assembly according to an embodiment of the present invention;

FIG. 16 illustrates a perspective view of a carriage apparatus, system, and method according to an alternative embodiment of the present invention;

FIGS. 17A-17C illustrate a perspective view, a partial view, and an exploded perspective view, respectfully, of a clamp assembly, according to an embodiment of the present invention;

FIGS. 18A-18D illustrate a perspective view, a left-side view, a back side view, and a front side view, respectively, of a track clamp, according to an embodiment of the present invention;

FIG. 19A illustrates a cross-sectional view taken from the line shown in FIG. 16 of the carriage apparatus, system, and method according to the alternative embodiment of the present invention;

FIG. 19B illustrates an enlarged cross-sectional view taken from the line shown in FIG. 19A thereof; and

FIG. 20 illustrates a perspective view of a locking element thereof.

### DETAILED DESCRIPTION

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention. For a better understanding of the present invention, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “adjustable cross nut” may refer to a removeable insert having internal threads that is installed into a non-threaded hole (also known as a through hole, or thru-hole); once installed, it is then possible to insert a bolt or threaded rod into the adjustable cross nut; the adjustable cross nut also has at least one “internal start”, positioned at a discrete radial location with respect to the central axis of the adjustable cross nut, where the cross nut may be adjusted such that the “internal start” is then positioned at a different discrete radial location with respect to the central axis of the adjustable cross nut.

As used herein the term “body” “body part” “extremity” or “limb” may refer to a body part or extremity consisting of foot, ankle, knee, leg, hand, wrist, arm and shoulder of a patient.

As used herein the term “clocking” or “clock” may refer to adjusting the radial location of the handle of a clamp, whereupon the handle reaches an adequately tightened position. For example, clocking may refer to an adjustable cross nut having at least one “internal start” that is moved radially from a first discrete radial location to a second discrete radial location and made lockable so that the internal start maintains the second discrete radial location when the system is subject to forces corresponding to a clamping action.

As used herein the term “flash” sterilization may be defined as steam sterilization of an unwrapped object at 132° C. for 3 minutes at 27-28 lbs. of pressure in a gravity displacement sterilizer as pioneered by the Underwood and Perkins. Currently, the time required for flash sterilization depends on the type of sterilizer and the type of item, i.e., porous vs non-porous items. Although the wrapped method of sterilization is preferred for the reasons listed below, correctly performed flash sterilization is an effective process for the sterilization of critical medical devices. Flash sterilization is a modification of conventional steam sterilization (either gravity, pre-vacuum, or steam-flush pressure-pulse) in which the flashed item is placed in an open tray or is placed in a specially designed, covered, rigid container to allow for rapid penetration of steam.

As used herein the term “holder” or “extremity holder” may refer to a device or a means for supporting the body part, or joint thereof, of a patient in the preparation for surgery or medical activity and/or during surgery to hold a body part for access to the body part. A holder can be used to position the body part such as an arm or leg for patient preparation.

As used herein the term “patient” may refer to any recipient of health care services. The patient is most often ill or injured and in need of treatment by a surgeon, physician, physician assistant, advanced practice registered nurse, veterinarian, or other health care provider.

As used herein the term “medical activity” may refer to the provision of medical care consisting of hospital activities, medical and dental practice activities, and “other human health activities” by a surgeon, physician, physician assistant, advanced practice registered nurse, veterinarian, or other health care provider. “Medical activity” can also include to numerous activities of medical treatment, diagnosis, preparation for surgery, or inspection of a body part so as to hold a body part for access to the body part.

As used herein the terms “surgery”, “operation”, or “surgical procedure” may refer an act of performing surgery such as by a surgeon, or simply to investigate and/or treat a pathological condition such as disease or injury, or to help improve bodily function or appearance using operative manual and instrumental techniques on a patient to in the medical specialty context. The adjective surgical may pertain to surgery; e.g., surgical instruments or surgical nurse. The patient or subject on which the surgery is performed can be a person or an animal.

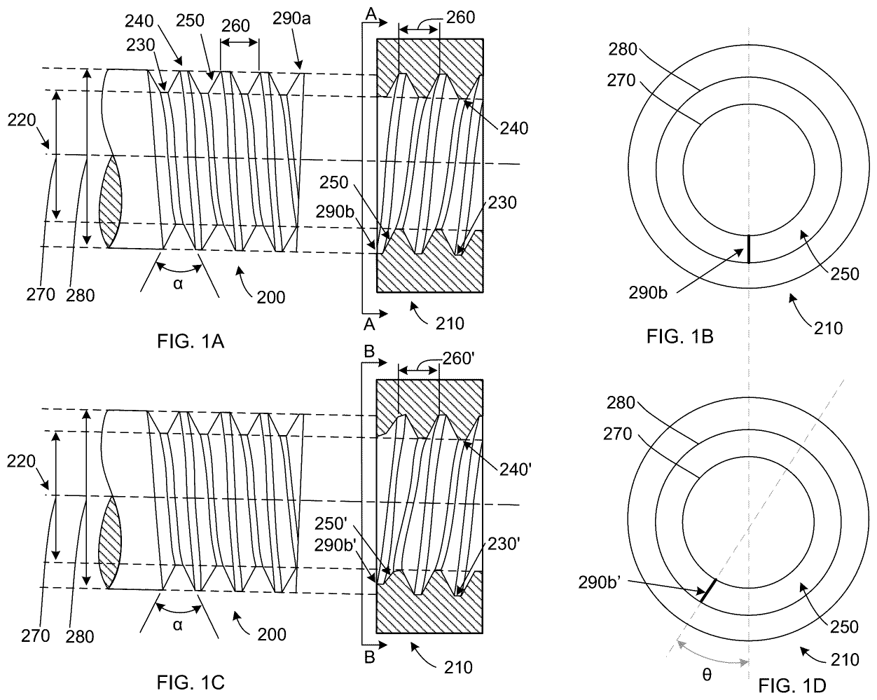

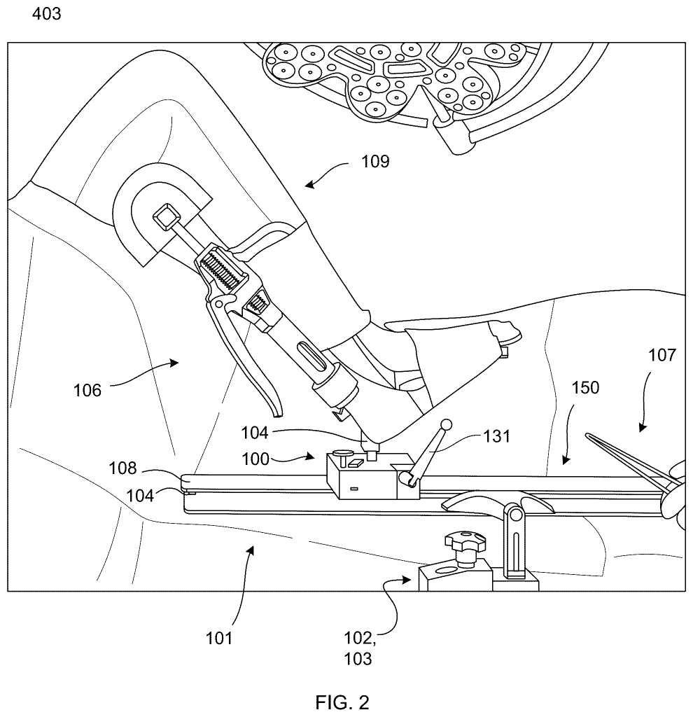

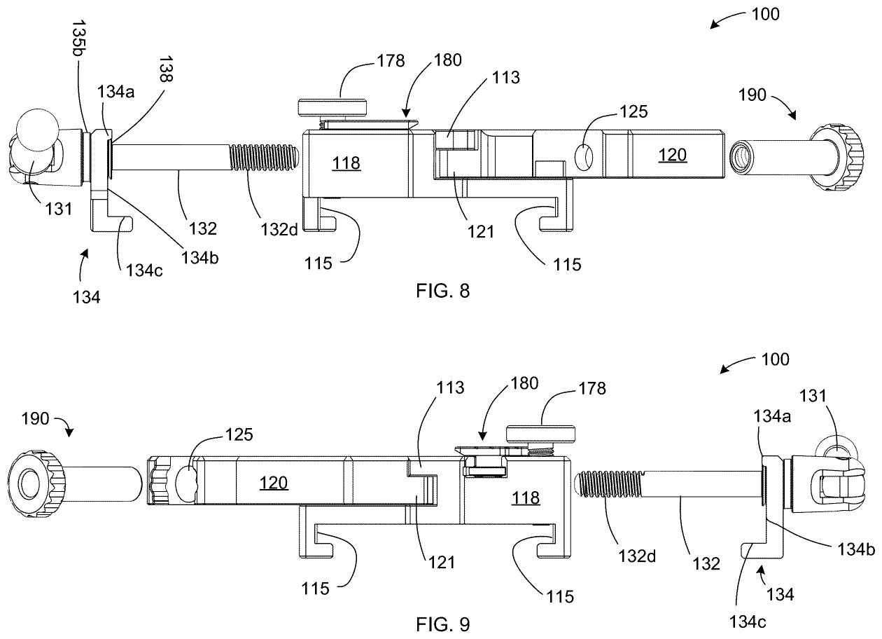

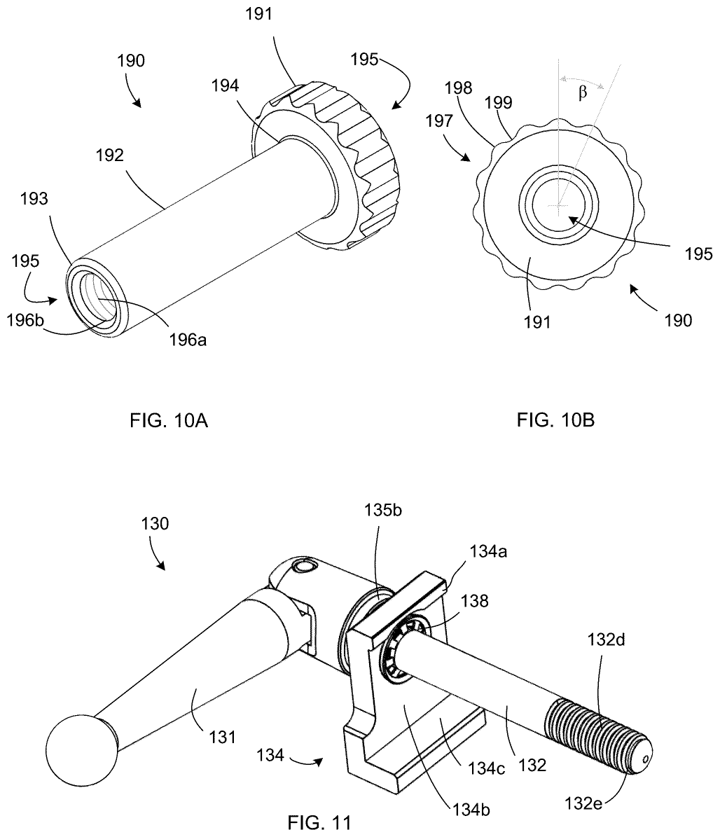

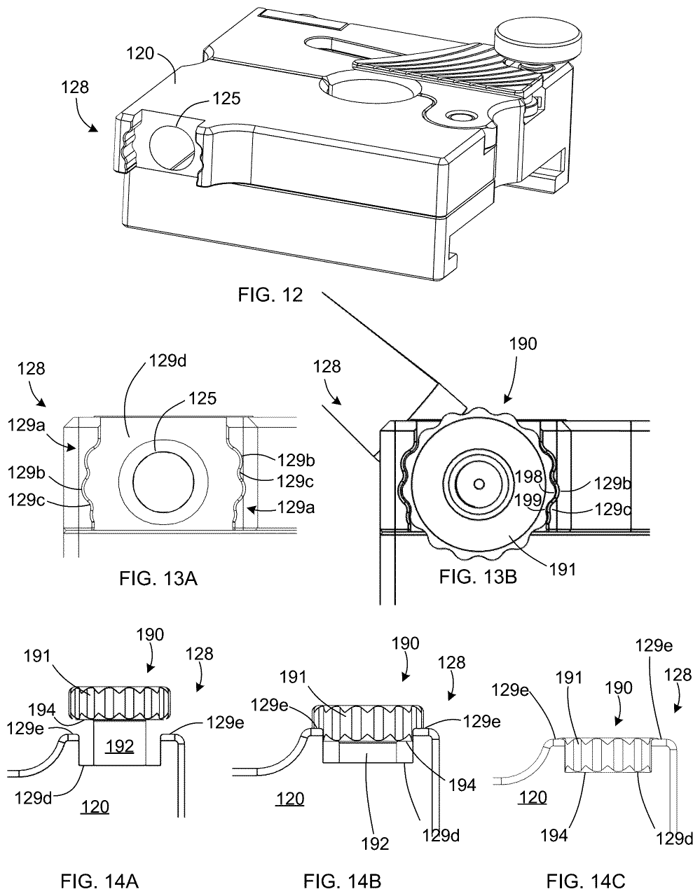

Referring to FIGS. 1A-1D through 20, an apparatus, system, and method of assembly of an apparatus for securing an extremity holder to a track secured an operating table useful for performing a surgical procedure using the apparatus, is generally shown as element 100; the carriage 100 is described in one or more exemplary embodiments of the present invention. FIG. 2 provides an exemplary, full-assembled arrangement of an extremity holder 106 coupled to an apparatus 100, where the carriage 100 may be operably attached to a track 108 of a base plate 150. Base plate 150 may be disposed on an operating room (OR) table 101 (shown in FIG. 2 with sterile covering), where base plate 150 is coupled to side rail clamp 103, which in turn, is clamped to a side rail 102 of OR table 101. Extremity holder 106 of a pre-determined shape may be attached to a post 104 at a proximate end and the post 102 may have, at a distal end, a ball (shown in FIG. 2) secured at least partially within carriage 100. In this way, carriage 100 may provide for simultaneous locking of two or more components—in the case of FIG. 2, an extremity holder 106 via a ball embedded within the carriage 100 and at a discrete location along track 108—achieved by one locking mechanism, partially shown in FIG. 2 at handle 131. The operating table 101 can have a track 108 as shown in FIG. 2, either integral or removably coupled to the operating table 101 for example, the track 108 may be integral to a base plate 150 secured to the operating table 101, for positioning the extremity holder 106 thereby positioning a body part 109 for the surgical procedure.

Referring to FIGS. 1A-1D through 20, the carriage 100 may comprise a carriage body 110, a handle assembly 130, and optionally an adjustable cross nut assembly 190, the carriage 100 adapted to support the extremity holder 106 on the track 108 and for selective movement of the carriage assembly 110 there along. The carriage assembly 110 may include a base 111 having a rigid rectangular structure with a step 112. The base 111 may comprise a hinge portion 113 having a first knuckle 113a, located at one end of the step 112 adapted to join an arm 120 thereto and may include a recess portion 114 located along said step 112 adapted to accept a protrusion 127 on arm 120. The base 111 may further comprise a track portion 115 defining rails to attach to the track 108, or to a track 108 operably connected by an extendable track thereof, such as that sold by Innovative Medical Products™ and taught by U.S. Pat. No. 11,839,574 B2, commonly owned by Applicant and hereby incorporated by reference.

The base 111 may include an upper surface 116, a lower surface 117 and side surfaces 118. The upper surface 116 may be configured with the step 112 generally located and centered approximately along a mid-line, the hinge portion 113 may be located at one end of the step 112, and the recess 114 may be located at another end of the step 112. The track portion 115 may be located on a lower surface 117 of the base 111. The base 111 may have side surface(s) 118 so as to define a rigid rectangular structure, which is non-limiting as other designs are possible. Because the base 111 may be deployed for use in an operating room environment, it may be formed from materials that are durable, sturdy, and that can be repeatedly sterilized such as, for example, most commonly formed from stainless steel that is known to reduce bacterial and early bio-film attachment, and/or other suitable metals and metal alloys. The base 111 may have an opening 119 in a side surface 118 configured to accept a rod 132 of a handle assembly 130 as is described herein.

The carriage 100 of the present invention may further comprise an arm 120 having a first arm side 120a having an arm hinge portion 121 with a second knuckle 121a and an opening 122 formed as a hole configured to operably receive a pin 123 to join the arm 120 to the 111. The arm 120 may be configured to fit to the base 111 adjacent the step 112 whereby the hinge portion or second knuckle portion 121a aligns with the hinge portion 113 or first knuckle 113a on the base 111 so as to be attached with pin 123, thereby opening and closing of the arm 120 by rotation around the pin 123. The first and second knuckle, 113a and 121a can include a sleeve bearing 124, or other known rotational joint type, to provide smooth rotation around the point formed by the hinge portions and pin 123. The arm 120 can be a general rectangular shape and formed from a solid sterilizable material. A suitable material to form the arm 120 is stainless steel yet other materials and/or metal alloys with qualities of durability, strength and that can be repeatedly sterilized to reduce bacterial attachment can be utilized.

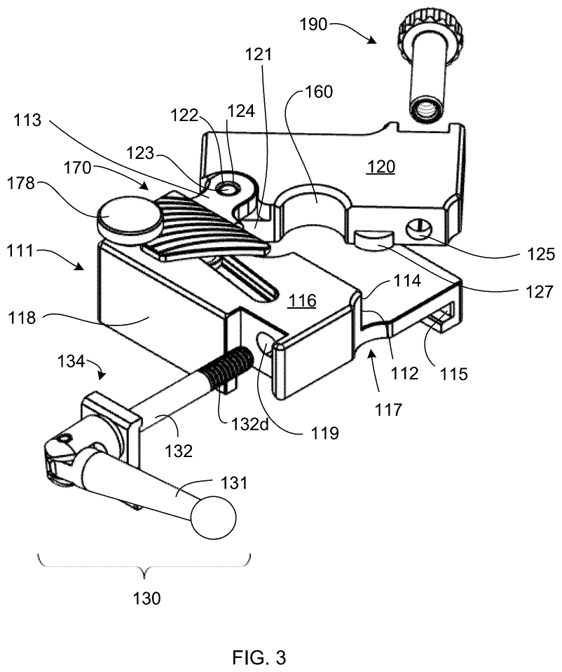

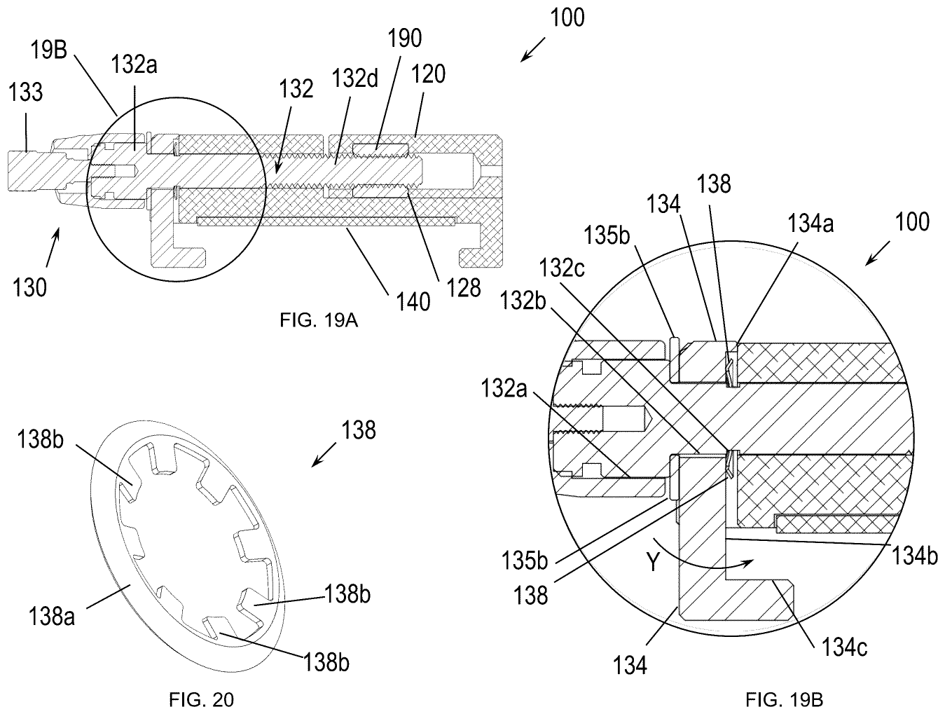

As shown in FIG. 3, the arm 120 may be configured with an opening 125 adapted to receive an adjustable cross nut 190, configured to tighten the handle assembly 130 and perform the holding, securing, and clocking feature(s) of the present invention. The adjustable cross nut 190, in conjunction with features of the carriage assembly 110, generally provide for clocking of the apparatus 100, as described in detail in Applicant's commonly-owned U.S. patent application Ser. No. 17/187,759, U.S. Pat. No. 12,150,818 B2. Alternatively, the receiver threads 190 may be fixedly coupled within the arm 120, such as shown in FIG. 19A. The arm 120 may be further configured with a protrusion 127 adapted to fit into the recess 114 in base 111 adjacent the step 112 by closing the arm 120. The arm 120 is further configured with a sleeve head receiver 128, disposed on a second arm side 120b, as will be further detailed below.

As shown in FIGS. 1A-1D through 20, the assembly 100 of the present invention is adapted to secure and to hold the extremity holder 106 in a desired position, thereby positioning a body part 109 for a desired surgical procedure 107. The carriage 110 may be configured with a socket 160 formed at a mid-point in the upper surface 116, adapted to receive a ball or other attaching means of the extremity holder 106. The socket 160 may be formed between the base 111 and arm 120 in the closed position. As the arm 120 abuts the step 112, the protrusion 127 may be inserted into the recess along the step 112, thereby creating strength and proper alignment with respect to the holding of the ball. The socket 160 can be tightened or loosened to open and close upon the ball by the clamping action of the clamp assembly 120. For example, a tightening operation involves turning the handle 131 in a particular direction which pulls the arm 120 by engaging a threaded portion 132d of the threaded insert 126 in the hole 125 of the arm 120 with the threaded portion 132d of rod 132. The base 111 and arm 120 may be joined to form a fulcrum at the hinge formed by the pin 123 and hinge portions 113, 121, thereby tightening the hold around the ball in the socket 160.

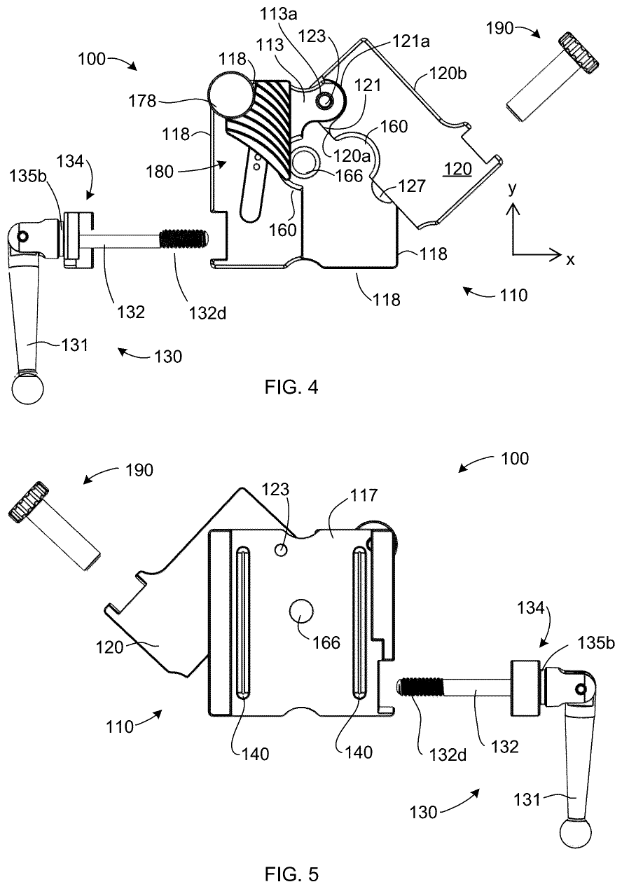

As seen herein in FIG. 4, hinge portions 113, 121 may be offset from the center line of socket 160, as well as the surface 121, and this offset may provide for a number of advantages. In a first advantage, offsetting the hinge allows for control of the shear forces between the ball and socket 160 during tightening. In this context, “offset” refers to, for example as shown in FIG. 4, offsetting the pin 113a in the x-direction from a line through the center of the socket 160, the line being drawn in the y-direction. The diameter of the socket 160 may be slightly larger than the ball such that contact between the two occurs at two points. Two-point contact may be preferable, as it may render the ball and socket joint more controllable when taking into account manufacturing tolerances. If the pin 113a aligns with the edge of the socket 160 (i.e., the hinge is offset from the centerline by one half the diameter of the socket 160), then as the arm 120 approaches the closed position (as the arm 120 approaches base 111), the motion of the arm socket at the point of contact will theoretically have an entirely x-component movement. If, however, the pin 123 is misaligned with the edge of the socket 160 (i.e., the hinge is offset from the centerline by more or less than one half the diameter of the socket 160), then the motion of the arm 120 as it advances toward the base 111 will have a slight y-component in addition to the large x-component. This will provide a shear force in the y-direction, causing an increase in static friction between the ball and socket 160, due to the formation of Van der Waals bonds. The second advantage of moving the pin 123 and hinge portions 113, 121 to an offset position, is that it makes room for the tilt control assembly 170.

Post-surgery, or for the purposes of repositioning, a loosening operation involves turning the handle 131 in a particular direction opposite the tightening direction, thereby releasing the hold around the ball in the socket 160 by the similar action of the threads pushing the arm 120 outwardly by the action of engaging the threads of the threaded portion 132d and adjustable cross nut assembly 190.

As shown in FIGS. 1A-1D through 20, a handle assembly 130 may be provided that solves, among other problem-solutions, the problem of proper clocking, so that the handle tightens in a configuration that is advantageous to the surgeon and optimal for proper use of the carriage 100, such as the handle 131 fully-tightened position shown in FIG. 2. With respect to proper clocking, this disclosure according to the present invention contemplates at least three distinct embodiments of the handle assembly 130, and at least two distinct embodiments of the receiver threads 190. Any of the various handle assembly 130 embodiments may be combined with any of the various receiver thread 190 embodiments to arrive at the present invention as contemplated herein.

In a first embodiment of the handle assembly 130, as shown in the design of handle assembly 130 of FIGS. 2-9, 11, 13B, 15A-15F, 17A-17C, 18A-18D, and 20, the clocking (i.e., the fully-tightened position and/or angle of the handle 131 relative to the carriage assembly 110) of the handle 131 may be adjusted by providing one or more washers 135b. A user-selected, predetermined thickness of the washer 135b will result in a specific angular position of the fully tightened handle 131. A first washer 135b may be replaced with a second washer 135b having a different thickness, so as to affect the clocking angle of the fully-tightened handle. As other parts wear, such as for example the receiver threads 190, the interchangeable washer 135b can be replaced. In a preferred embodiment thereof, and as described herein, a locking element 138 may be semi-permanently coupled to the rod 132, so that a tab 134 and one or more washers 135b remain operably coupled to handle assembly 130, unless and until the carriage 100 wears out sufficiently, at which point the carriage 100 may be sent to the manufacturer for retooling. Retooling in this manner may require destroying, or otherwise removing, the locking element 138 and decoupling the tab 134 and existing washer(s) 135b, and then selecting a new washer(s) 135b having a user-selected, predetermined thickness. The design of the existing washer(s) 135b and the replacement new washer(s) 135b may be of different thicknesses with respect to one another, so that the rotational angle of the tightened clamp assembly varies by an appropriate amount and according to the amount of wear, so that the tightened position ends up in an optimal rotational angle, for example, between approximately a twelve-o'clock and approximately a three-o'clock position; or more preferably between about a twelve-thirty and a two-o'clock position; or most preferably about a one-o'clock position. This arrangement provides added benefits of keeping the components together so that they aren't lost in the hospital, such as in between surgeries. Another characteristic of this embodiment is that the handle 131 may rotate relative to the rod 132 to flip about the rod 132 by approximately 180 degrees, as is described in detail below.

In a second embodiment of the handle assembly 130 according to the present invention, the function of the first embodiment is similarly provided, except that in the second embodiment, the handle 131 is rigidly coupled or attached to the rod 132.

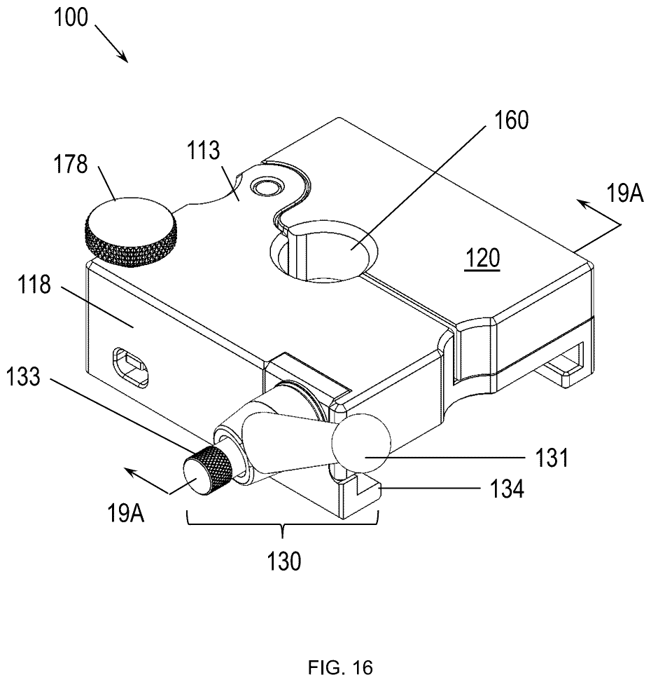

In a third embodiment of the handle assembly 130, an adjustable handle may be provided according to the present invention. A handle assembly 130 may include an adjustable handle 131 as shown, for example, in FIGS. 16 and 19A-19B. Handle assembly 130 may comprise an adjustable handle 131 having handle teeth, a rod 132 having rod teeth extending from a rod head 132a, a handle fastener 133 operably coupling the adjustable handle 131 to the rod head 132a, and a biasing element disposed between the handle fastener 133 and the adjustable handle 131. In operation, the handle fastener 133 may be hand-actuated or driven by a screwdriver or other tool, to adjust the handle 131 and its position on the threaded rod 132 to which it is attached. In this embodiment, clocking may be achieved by loosening the handle fastener 133, changing the orientation of the handle 131 relative to the rod 132, and reengaging teeth, or other rotational-prohibiting features upon retightening of the handle fastener 133. A biasing element, such as spring-loaded component (not shown) may further facilitate one-handed operation of the handle assembly 130 during the re-clocking procedure. An adjustable handle 131 may be made for example from casted stainless steel, such as Part No. 98258SP available from Monroe Engineering. The design according to the present invention in the third embodiment is a handle assembly 130 that may be adjusted incrementally to provide optimal clocking angle when the handle 131 is in the fully-tightened position, as illustrated for example in FIG. 2.

With respect to the receiver thread 190 embodiments, in a first embodiment, the present invention may provide an adjustable cross nut 190 to achieve modifiable clocking with reference to Applicant's commonly-owned U.S. patent application Ser. No. 17/187,759, U.S. Pat. No. 12,150,818 B2. This design is represented in the handle assembly 130 and also in the adjustable cross nut assembly 190 of FIGS. 3-9, 13B, 15A-15F, 17A-17C, 19A-19B and 20. This arrangement of parts may have the added benefit of onsite adjustment, for example, executed by the surgeon or surgical staff before and/or during surgery. Consequently, the second embodiment also may use adjustable washer(s) 135b of the first embodiment selected by hospital personnel so that correction of the carriage 100 may be achieved onsite.

In a second embodiment, the receiver threads 190 may be rigidly affixed or coupled to the carriage arm 120, as represented in the rigidly coupled receiver threads 190 of FIG. 19A. The receiver threads 190 may be tapped directly into the carriage arm 120, or alternatively, the receiver threads 190 may be a tapped cylindrical insert 190 formed of an advantageous material (e.g., harder or more durable material) that is then rigidly coupled with the carriage arm 120. In an alternative second embodiment, receiver threads 190 may be removeable from the carriage arm 120, via a carriage arm receiver thread opening 128, as illustrated in FIG. 19A, or other opening. A removeable receiver thread 190 and opening 128 may provide for better cleaning in between uses.

Other embodiments having the functional description of the clocking mechanism of the handle assembly 130 and/or the adjustable cross nut assembly 190 are considered to be within the scope of this disclosure.

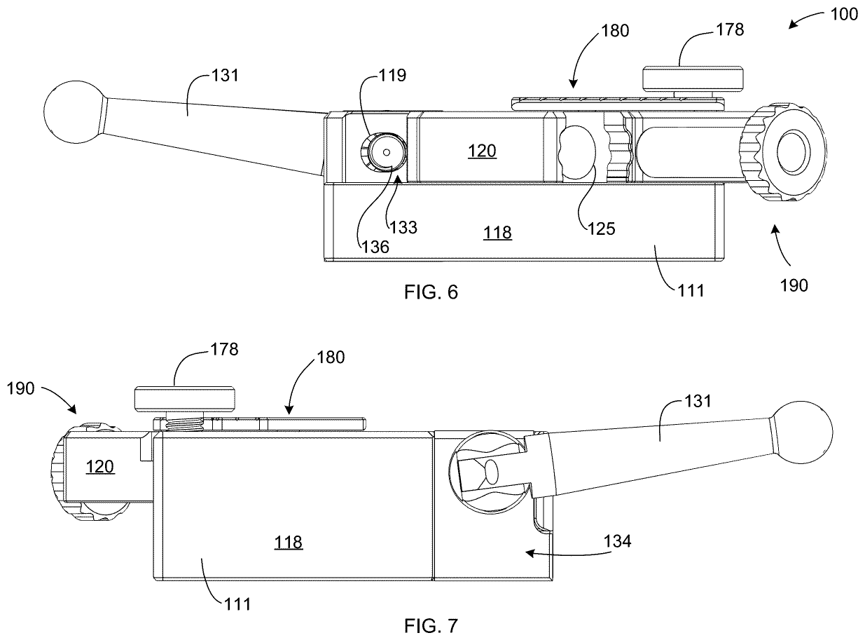

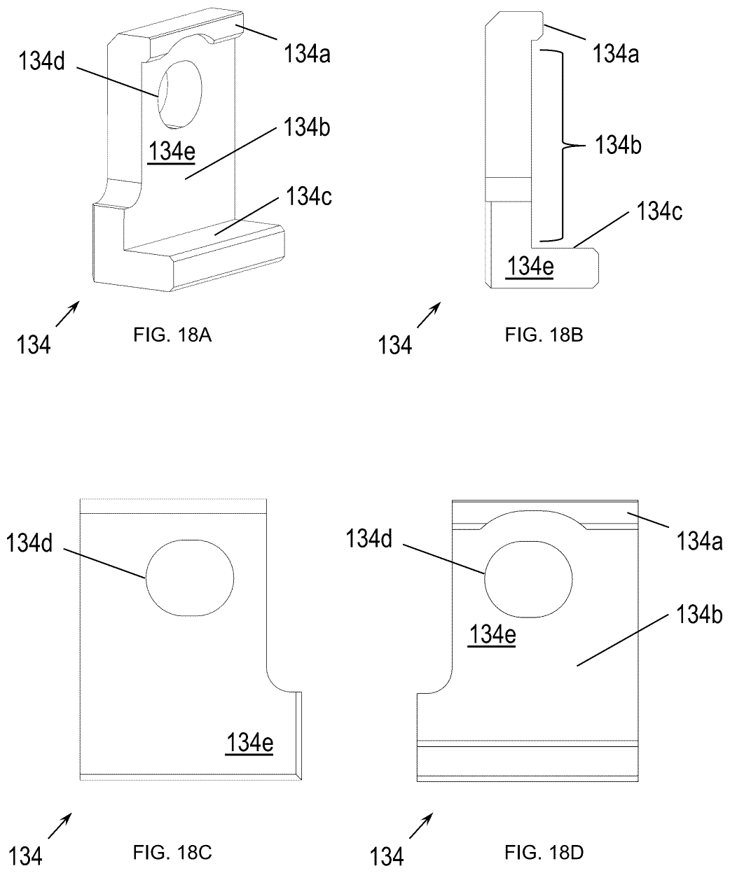

Regarding the various embodiments of the handle assembly 130, and as appropriate, the handle assembly 130 may comprise a handle 131, a rod 132, a tab 134, a retaining washer 138, and a washer 135b. The handle 131 may be made from stainless steel and have an ergonomic design suitable to provide tightening and improved operation in a user's hands. The rod 132 may similarly be made from stainless steel, and may have a rod threaded portion 132d having an external thread start 132e. Rod 132 may be configured to be received within opening 119 as demonstrated in at least FIGS. 3-9. Tab 134 may also be made of stainless steel, and the washer 135b may be made of PEEK material; polyether ether ketone (PEEK). As provided in FIG. 6—specifically with reference to opening 119—opening 119 may take the form of a slotted opening to facilitate proper aligning of rod 132 and adjustable cross nut assembly 190. Similarly, tab 134 may have a slotted opening 134d, as shown in FIGS. 18C-18D, whereby these slotted openings 119, 134d aid in registering the slotted opening 134d and rod 132 with the receiving end of element 190, as arm 120 and body 111 are naturally misaligned when the arm 120 is in an open, or even a slightly open, position.

Regarding the rod threaded portion 132d, there may be a single external thread start 132e that may include the geometric parameters as shown in FIG. 1A-1D, where (among other components) rod thread start 132e corresponds to thread start 290a. Alternatively, apparatus 100 may use thread structure 132e that employ a plurality of thread starts. Clockwise or counterclockwise threads 132d are considered within the scope of this disclosure.

As shown in FIGS. 11 and 18A-18D, tab 134 may comprise a body 134e including a first tab clamp portion 134a extending outwardly from the body 134e, a second tab clamp portion 134b formed by at least a portion of the body 134e, a tab track portion 134c extending outwardly from the body 134e, and an opening 134d extending through the body 134e. The first tab clamp portion 134a and second tab clamp portion 134b may be suitable for clamping of two separate components, quasi-independently, independently, via a single hand motion, and/or simultaneously, with respect to one another. The first tab clamp portion 134a may be suited to provide a clamp action of an extremity holder 106 used to provide the tab 134 with the ability to pivot to facilitate locking of the carriage 100 onto the track 108. The second tab clamp portion 134b, in a preferred embodiment, is offset from the first tab clamp portion 134a in the manner shown in FIG. 18B; this offset is what may allow the tab 134 to pivot about the first tab clamp portion 134a when the handle assembly 130 is in an assembled configuration and then is tightened. The tab track portion 134c may be provided to keep the carriage 100 from lifting upwardly and away from the track 108, or from sliding off of the track 108; the latter benefit may be aided and/or accomplished through the use of a spring pin 104 (see FIG. 2), which may be configured to abut tab track portion 134c, and/or other portions of the tab 134.

FIGS. 19A-19B and 20 illustrate the functionality of the tab 134, in addition to disclosing other features. In FIG. 19A, a cross-sectional view, taken through the line shown in FIG. 16 is shown. This illustrates one of the various embodiments taught by the instant invention, with respect to handle assembly 130 and adjustable cross nut assembly 190. In FIG. 19B, an enlarged cross-sectional view taken from the line shown in FIG. 19A is provided. The track clamp assembly 134 is, upon tightening of the handle 131 of the handle assembly 130, adapted to first make contact at the first tab clamp portion 134a of the base 111 (i.e., the hatched region proximate 134a as shown in FIG. 19B) to provide secure coupling of the extremity holder 106 to the carriage 100. At this point, the tab 134 may be positioned approximately vertical, as shown in FIGS. 19A-19B, with respect to (or orthogonal with respect to) the base 111. Upon further tightening of the handle 131 of handle assembly 130, the first tab clamp portion 134a acts as a pivot point, a pivot surface, or a pivot line, and the lower portion can swing in the direction toward the track 108, denoted as “Y” in FIG. 19B. This makes further tightening possible of the rod 132 and ultimately brings the second tab clamp portion 134b to fixedly couple to the adjacent surface of the base 111 to thereby provide complete clamping action of the handle assembly 130 and the carriage 100 with respect to both components to be clamped, e.g., the extremity holder 106 and the track 108 (track 108 is not shown in FIG. 19B, but is shown in FIG. 2) of the base plate 150. In effect, this ultimately achieves quasi-independent clamping, multi-action clamping, or equalizing clamping, and the tab 134 in this sense may act as a balancer to provide clamping of the first component while allowing incremental moving of the second component and then subsequent rigid clamping, or coupling, of the second component. This is largely possible via the offset, or hollowed second tab clamp portion 134b, and with the clearances that are provided. In alternative embodiments according to the present invention and considered as falling within the scope of this disclosure, it is conceived to have a carriage 100 wherein the order of clamped components is reversed, i.e., the track 108 is clamped first, then the extremity holder 106, via a tab 134 having a different arrangement of parts.

Additionally, the handle assembly 130 may further comprise one or more locking elements 138, which may be a locking element, such as an external ring retainer. The locking element 138 may be coupled to the rod 132 proximate a rod tab receiver 132b to provide retention of the tab 134, coupled proximate the rod tab receiver 132b, and/or the washer 135b, coupled proximate the track assembly 134 and/or the rod tab receiver 132b. The arrangement of these parts provides for appropriate tolerances so that the components can move relative to one another; e.g., to allow the handle assembly 130 to properly rotate relative to base 111 to provide simultaneous, controlled clamping of both components, e.g., extremity holder 106 and track 108 of base plate 150.

The locking element 138, as shown in FIG. 20, may include a first locking portion 138a and a second locking portion 138b. As illustrated in FIG. 19B, locking element 138 may serve a primary purpose of retaining the track clamp assembly 134 and/or the washer 135b. Additionally, the locking element 138 may provide a biasing action upon being compressed, so as to deflect and provide the necessary space for the track assembly 134 to couple and clamp e.g., the track 108 not shown in FIG. 19B, but shown in FIG. 2) of the base plate 106, while also operably coupling handle assembly 130 components so that they move relative to each other as purposed, but do not come apart. Locking element 138 may advantageously keep handle assembly 130 components together so that hospital personnel don't lose the various essential elements.

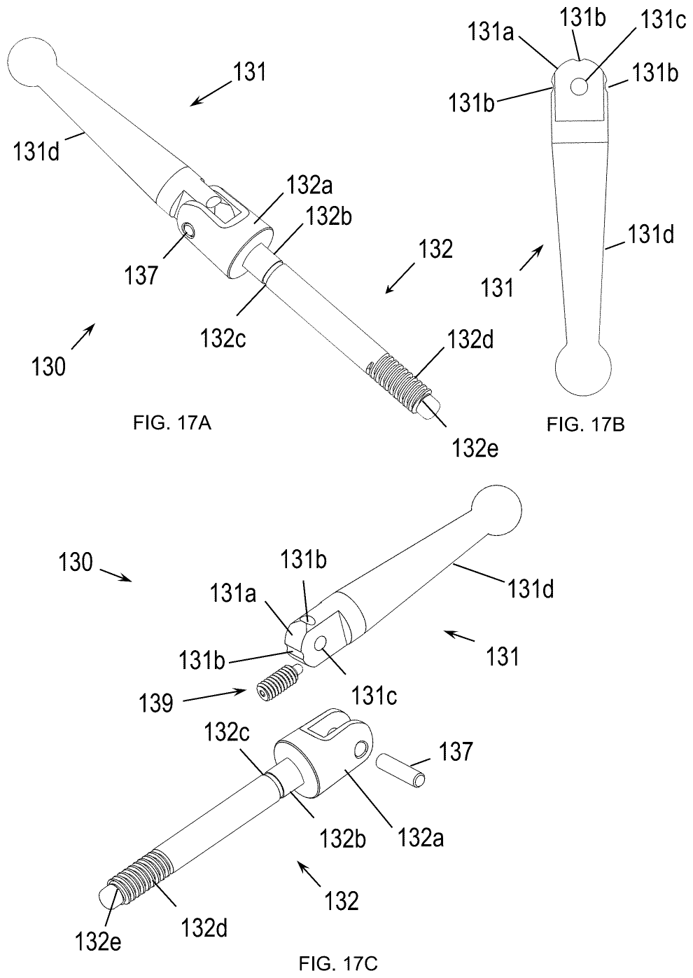

Referring to the embodiment(s) of the handle assembly 130 illustrated in FIGS. 17A-17C, certain aspects of the present invention are disclosed. Handle assembly 130 may comprise a handle 131, a rod 132, and a pin 137 coupling said components about a pivotable axis of rotation. As shown in FIG. 17C, handle 131 may include an arcuate surface 131a, one or more depressions 131b disposed therealong, a handle opening 131c adapted to receive the pin 137, and a handle grip 131d adapted for operable movement by surgical staff. The rod 132 may include a rod head 132a, a rod track clamp receiver 132b a rod locking element receiver 132c, a rod threaded end 132d, and a rod thread start 132e. The rod head 132a may include an opening for receiving the pin 137. A spring plunger 139 may be threadedly inserted into the rod head 132. Spring plunger 139, or pogo pin, may be a component that is a spring action pin, upon being threaded into a corresponding component, wherein the pin translates in the axial direction. Spring plunger 139 may be biased open in a free-standing state, and may be biased closed when acted upon by an external force. In operation, and in an assembled configuration, the handle 131 may be rotated relative to the rod 132 so that the spring plunger 139 is biased open when moved proximate a depression 131b, and is biased closed when moved along and proximate the arcuate surface 131a. This achieves a desired effect of keeping the handle 131 in a relatively stationary position as compared to the rod 132, when the clamp assembly is actively being adjusted. Advantageously, the present invention may include the handle assembly 130 wherein the handle 131 remains stationary relative to rod 132 when handle assembly 130 is in a partially threaded, or fully tightened position. For example, the handle assembly 130 of FIGS. 17A-17C provide for approximately a 180 angular degree swing of handle 131d relative to rod 132. This flip handle arrangement provides for continual rotation of the handle 131d, when in an assembled configuration as shown in FIG. 2, wherein the base plate 106 may inhibit true 365-degree, continuous rotation.

As shown in FIG. 5, for example, at least one sliding plate 140 may be located between a lower surface 117 of the base 111 and the track 108 for improving sliding movement and positioning of said carriage 110 along the track 108. The at least one sliding plate 140 may be formed of a predetermined friction-reducing material, including a polymer and other materials that can be sterilized. The sliding plate 140 may be formed as one or more linear strips aligned length-wise to the sliding direction of apparatus 100 along track 108. Alternatively, sliding plate 140 may be formed as a generally planar surface, having a predetermined shape and having projections proximate an edge including protrusions that are configured for placement in openings of the base 111. In the alternative embodiment, the sliding plate 140 is configured to pivot between a closed position in a recess 114 in the lower surface 117 of the base 111 when interposed between the base 111 and the track 108 for improving sliding movement and positioning of said carriage 110 along the track 108. The sliding plate 140 then may be pivoted via the protrusions to an open position having the appearance of a flap useful for cleaning and sterilization as well as ease of maintenance and replacement of the sliding plate 140, while also providing for a retention mechanism that keeps the sliding plate 140 from being lost by hospital personnel.

Referring now to, for example, FIGS. 3 and 4, carriage assembly 110 may further comprise a tilt control assembly 170. The tilt control assembly 170 may include a slide assembly 180 configured to follow a pathway located on the base 111, formed with dimples, which trajectory and dimples can provide predetermined tilt angle(s). According to another aspect of the present invention, a knurled knob 178 may be formed in tilt control assembly 170 to tighten the carriage base 111 to an operating table 106 and prevent movement in all x, y, and z directions. The knurled knob 178 may be configured to be inserted into a threaded hole in the base 111 so as to set a desired location on the operating table 101, or other track. The knurled knob 178 is configured to lock carriage assembly 100 base 111 anywhere on track thereby forming an “e-brake” and stopping any translational force to assist the handle assembly 130 with additional locking so as not slide along the rail or track. These parts are minimized for sterilization as the knurled knob 171 requires removal and the hole and knob 171 can be easily cleaned and sterilized.

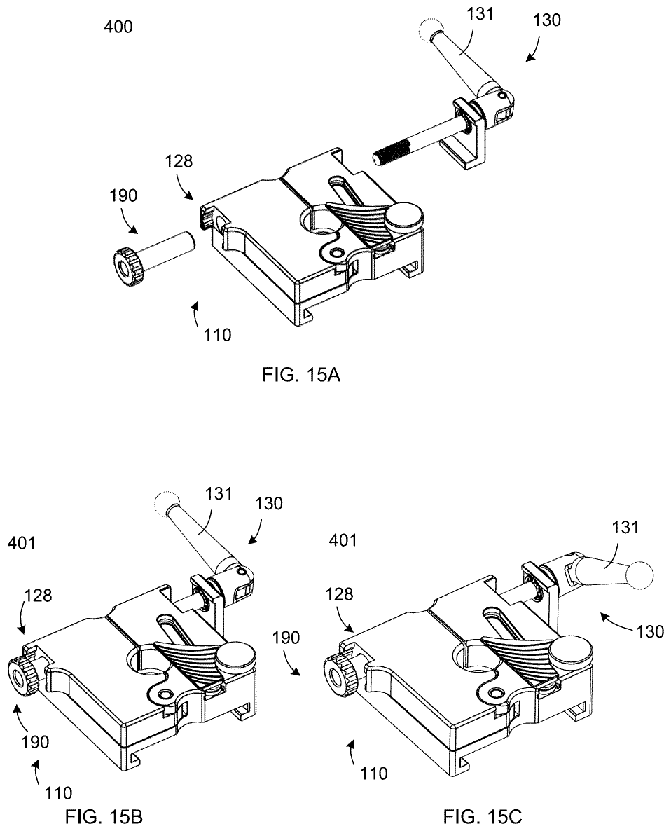

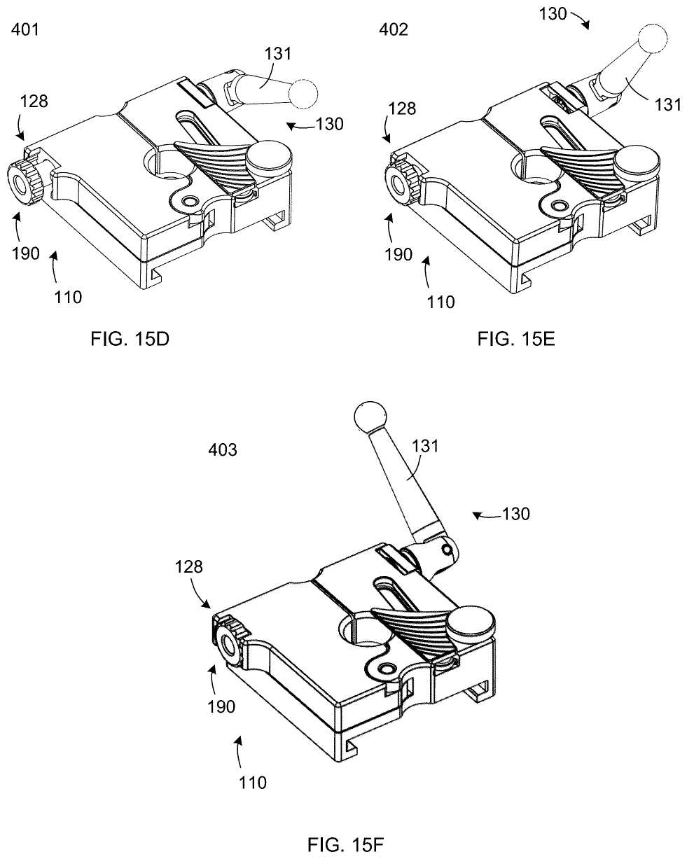

Referring to FIGS. 15A-15F, a method of assembling a carriage 100 is described, purposed for securing an extremity holder to a track 108 on an operating table 101 useful for performing surgical procedures. This procedure is disclosed in detail in Applicant's commonly-owned U.S. patent application Ser. No. 17/187,759, U.S. Pat. No. 12,150,818 B2 but illustrates an assembly process 400-403 of an embodiment(s) of the handle assembly 130 and/or adjustable cross nut assembly 190 that is helpful, and or analogous, to certain other embodiments of said components described herein.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A handle assembly for coupling one or more first surgical devices to one or more second surgical devices, the handle assembly comprising:
a handle for operable adjustment by a surgical personnel;
a rod adapted to couple to the handle, the rod including a rod head disposed at or proximate a first end, and a threaded portion disposed along a second end; and
a tab adapted to operably couple to the rod proximate the rod head, the tab including:
a tab body,
a first tab clamp portion extending outwardly from and along a first tab end of the tab body, and
a second tab clamp portion formed by at least a portion of the tab body, the second tab clamp portion disposed inwardly relative to the first tab clamp portion,
wherein, in an assembled configuration, the rod of the handle assembly extends through a first body so that the threaded portion of the rod is operably coupled to receiver threads disposed within a second body, the first body being pivotably coupled to the second body,
wherein, when the handle assembly is tightened:
in a first action, the first tab clamp portion of the tab couples to a side surface of the first body while moving the second body toward the first body to thereby clamp the one or more first surgical devices in between the first and second bodies, and
in a second action, the tab pivots about the first tab clamp portion in the Y direction, such that the second tab clamp portion of the tab moves toward a track portion of the first body to thereby clamp the one or more second surgical devices in between the second tab clamp portion and the track portion of the first body, to achieve simultaneous, multi-action clamping of said one or more first and said one or more second objects.

2. The handle assembly of claim 1, wherein the receiver threads within the second body comprise either rigidly coupled receiver threads in the second body, or receiver threads disposed in an adjustable cross nut assembly coupled to the second body.

3. The handle assembly of claim 2 further comprising:
a fastener adapted to pivotably couple the handle and the rod; and
a spring plunger operatively coupled between the handle and the rod head,
the handle further comprising an arcuate surface and a plurality of depressions, each depression characterizing a discrete handle position, the arcuate surface and plurality of depressions being disposed around a handle opening, the handle opening configured to receive the fastener,
wherein, upon pivoting the handle relative to the rod, the spring plunger and the plurality of depressions provide a holding force that retains each discrete handle position, when pivoted to that discrete handle position, thereby keeping the handle in a selected discrete handle position when the surgical personnel removes their hand from the handle.

4. The handle assembly of claim 3 further comprising:
a spacing washer characterized by a user-defined thickness, disposed along the rod between the rod head and the tab, the spacing washer suitable for providing a desirable clocking angle of the handle assembly, when the handle is in a fully tightened position subsequent the second action.

5. The handle assembly of claim 4 further comprising:
a locking element operably coupled to the rod along a rod locking element receiver, the locking element disposed proximate the tab, the tab being operably coupled to the rod along a rod tab receiver,
wherein the locking element inhibits the tab from being removed from the handle assembly, while also being adapted to bias flat when the first and/or second actions are achieved by tightening of the handle assembly.

6. The handle assembly of claim 2, wherein the handle assembly comprises the handle rigidly coupled to the rod.

7. The handle assembly of claim 2, wherein the handle assembly comprises an adjustable handle including a handle fastener and a plurality of rotational prohibitors, and the rod head comprises a plurality of corresponding rotational prohibitors, such that adjustable clocking is achieved by loosening the handle fastener, rotating the handle relative to the rod, and retightening the handle fastener.

8. The handle assembly of claim 2, wherein the first body comprises a carriage base, the second body comprises a carriage arm, the one or more first surgical devices comprises an extremity holder, and the one or more second surgical devices comprises a track of a base plate and/or a side rail of a support table.

9. The handle assembly of claim 8, wherein the tab further comprises a tab track portion extending outwardly from and along a second tab end of the tab body, for coupling underneath the track of the base plate to thereby inhibit the carriage base from lifting off of the track.

10. The handle assembly of claim 9, wherein the carriage base and carriage arm comprise a socket joint, and wherein the extremity holder comprises a ball, such that in an assembled configuration, a ball-and-socket joint is formed.

11. The handle assembly of claim 10, wherein when the handle assembly achieves the first action, the extremity holder becomes rigidly coupled to the carriage base and the carriage arm, while the carriage base remains slidably coupled to the track of the base plate.

12. A handle assembly for rigidly and/or operably coupling a body part of a surgical patient to a support table, the handle assembly comprising:
a handle for operable adjustment by a surgical personnel;
a rod adapted to couple to the handle, the rod including a rod head disposed at or proximate a first end, and a threaded portion disposed along a second end; and
a tab adapted to operably couple to the rod proximate the rod head, the tab including:
a tab body,
a first tab clamp portion extending outwardly from and along a first tab end of the tab body, and
a second tab clamp portion formed by at least a portion of the tab body, the second tab clamp portion disposed inwardly relative to the first tab clamp portion,
a carriage assembly including a carriage arm pivotably coupled to a carriage body, the carriage assembly configured to translate on a track of a base plate coupled to the support table,
wherein, in an assembled configuration, the rod of the handle assembly extends through the carriage body so that the threaded portion of the rod is operably coupled to receiver threads disposed within the carriage arm, and
wherein, when the handle assembly is tightened:
in a first action, the first tab clamp portion of the tab couples to a side surface of the carriage body while moving the carriage arm toward the carriage body to thereby clamp an extremity holder coupled to the body part in between the carriage body and the carriage arm, and
in a second action, the tab pivots about the first tab clamp portion in the Y direction, such that the second tab clamp portion of the tab moves toward a track portion of the carriage body to thereby clamp the carriage assembly to the track portion of the base plate, to achieve simultaneous, multi-action clamping of said one or more first and said one or more second objects.

13. The handle assembly of claim 12, wherein the receiver threads within the carriage arm comprise either rigidly coupled receiver threads, or receiver threads disposed in an adjustable cross nut assembly coupled to the carriage arm.

14. The handle assembly of claim 13 further comprising:
a fastener adapted to pivotably couple the handle and the rod; and
a spring plunger operatively coupled between the handle and the rod head,
the handle further comprising an arcuate surface and a plurality of depressions, each depression characterizing a discrete handle position, the arcuate surface and plurality of depressions being disposed around a handle opening, the handle opening configured to receive the fastener,
wherein, upon pivoting the handle relative to the rod, the spring plunger and the plurality of depressions provide a holding force that retains each discrete handle position, when pivoted to that discrete handle position, thereby keeping the handle in a selected discrete handle position when the surgical personnel removes their hand from the handle.

15. The handle assembly of claim 14 further comprising:
a spacing washer characterized by a user-defined thickness, disposed along the rod between the rod head and the tab, the spacing washer suitable for providing a desirable clocking angle of the handle assembly, when the handle is in a fully tightened position subsequent the second action.

16. The handle assembly of claim 15 further comprising:
a locking element operably coupled to the rod along a rod locking element receiver, the locking element disposed proximate the tab, the tab being operably coupled to the rod along a rod tab receiver,
wherein the locking element inhibits the tab from being removed from the handle assembly, while also being adapted to bias flat when the first and/or second actions are achieved by tightening of the handle assembly.

17. A handle assembly for securing a socket joint disposed in a carriage assembly configured to operably couple to a ball of an extremity holder for a surgical procedure, and to operably couple to a track of a base plate, the handle assembly comprising:
a handle for operable adjustment by a surgical personnel;
a rod adapted to couple to the handle, the rod including a rod head disposed at or proximate a first end, and a threaded portion disposed along a second end; and
a tab adapted to operably couple to the rod proximate the rod head, the tab including:
a tab body,
a first tab clamp portion extending outwardly from and along a first tab end of the tab body, and
a second tab clamp portion formed by at least a portion of the tab body, the second tab clamp portion disposed inwardly relative to the first tab clamp portion,
wherein, in an assembled configuration, the rod of the handle assembly extends through a carriage body of the carriage assembly so that the threaded portion of the rod is operably coupled to receiver threads disposed within a carriage arm of the carriage assembly, the carriage arm being pivotably coupled to the carriage body,
wherein, when the handle assembly is tightened:
in a first action, the first tab clamp portion of the tab couples to a side surface of the carriage body while moving the carriage arm toward the carriage body to thereby clamp the ball of the extremity holder within the socket joint, and
in a second action, the tab pivots about the first tab clamp portion in the Y direction, such that the second tab clamp portion of the tab moves toward the track portion of the carriage body to thereby clamp the track in between the second tab clamp portion and the track portion of the carriage body, to achieve simultaneous, multi-action clamping of said one or more first and said one or more second objects.

18. The handle assembly of claim 17, wherein the receiver threads within the carriage arm comprise either rigidly coupled receiver threads, or receiver threads disposed in an adjustable cross nut assembly coupled to the carriage arm.

19. The handle assembly of claim 18 further comprising:
a fastener adapted to pivotably couple the handle and the rod; and
a spring plunger operatively coupled between the handle and the rod head,
the handle further comprising an arcuate surface and a plurality of depressions, each depression characterizing a discrete handle position, the arcuate surface and plurality of depressions being disposed around a handle opening, the handle opening configured to receive the fastener,
wherein, upon pivoting the handle relative to the rod, the spring plunger and the plurality of depressions provide a holding force that retains each discrete handle position, when pivoted to that discrete handle position, thereby keeping the handle in a selected discrete handle position when the surgical personnel removes their hand from the handle.

20. The handle assembly of claim 19 further comprising:
a spacing washer characterized by a user-defined thickness, disposed along the rod between the rod head and the tab, the spacing washer suitable for providing a desirable clocking angle of the handle assembly, when the handle is in a fully tightened position subsequent the second action, and
a locking element operably coupled to the rod along a rod locking element receiver, the locking element disposed proximate the tab, the tab being operably coupled to the rod along a rod tab receiver,
wherein the locking element inhibits the tab from being removed from the handle assembly, while also being adapted to bias flat when the first and/or second actions are achieved by tightening of the handle assembly.
